(19) United States
(12) Patent Application Publication
(10) Pub. No.: US 2006/0058275 A1
(43) Pub. Date: Mar. 16, 2006

(54) Processes for preparing stabilized, highly pure rocuronium bromide

(76) Inventors: Oded Friedman, Talmey Yechiel (IL); Oded Arad, Rechovot (IL); Iosef Manascu, Omer (IL); Tamir Fizitzki, Beer-Sheva (IL); Joseph Kaspi, Givatayim (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 11/180,715

(22) Filed: Jul. 14, 2005

Related U.S. Application Data
(60) Provisional application No. 60/587,901, filed on Jul. 15, 2004.
(60) Provisional application No. 60/587,900, filed on Jul. 15, 2004.

(51) Int. Cl. A61K 31/58

(52) U.S. Cl. 514/176

(57) ABSTRACT

Processes are provided herein for the preparation and purification of stable, powdered solids comprising substantially pure rocuronium bromide.

### RELATED PATENT APPLICATIONS

The present application claims the benefit of U.S. Provisional Application No 60/587,901, filed Jul. 15, 2004, and U.S. Provisional Application No. 60/587,900, filed Jul. 15, 2004, the contents of which are herein incorporated by reference.

### FIELD AND BACKGROUND OF THE INVENTION

Neuromuscular blocking agents (such as, tubocurarine chloride, pancuronium bromide, vecuronium bromide, rocuronium bromide, atracurium besylate) are compounds with the similar muscle paralyzing activity as the alkaloid curare or d-tubocurarine. Neuromuscular blocking agents (NMBAs) interrupt transmission of nerve impulses at the skeletal neuromuscular junction.

Based on their mechanism of action, NMBAs are divided into two categories: noncompetitive depolarizing and competitive non-depolarizing NMBAs. Both NMBA types prevent acetylcholine from triggering the muscle contraction, hence are used as anesthesia adjuvants in the operating theatre for aiding intubation i.e. relaxation of vocal cords, jaw muscles etc. and also for surgery i.e. providing generalized muscle relaxation, as relaxants during electroshock, in convulsive states, etc. Typically, therapy is performed by i.v. administration of a suitable dosage form.

1-[(2β,3α,5α,16β,17β)-17-acetoxy-3-hydroxy-2-(4-morpholinyl)-androstan-16-yl-]-1-(2-propenyl)pyrrolidinium bromide, also known by the name rocuronium bromide, is a steroidal neuromuscular blocking agent having the structural formula

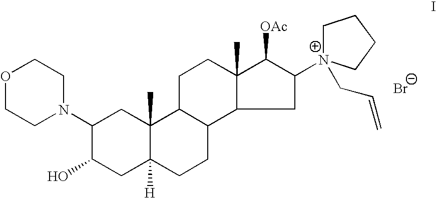

Presently, rocuronium bromide is available commercially under the brand names Esmeron® and Zemuron®,

Rocuronium bromide and the intermediates thereof were described in U.S. Pat. No. 4,894,369 to Sleigh et al. and generally in a paper by Zoltan et al. Current Medicinal Chemistry, 9(16), 1507-1536, 2002, which are incorporated by reference as if fully set forth herein. Accordingly it is obtained by reacting 2-propenyl bromide with (2β,3α,5α,16β,17β)-2-(4morpholinyl)-16(1-pyrrolidinyl)-androstane-3,17-diol 17-acetate having the structural formula (II) in dichloromethane, followed by column chromatography and precipitation of the pure product from a mixture of dichloromethane and diethyl ether.

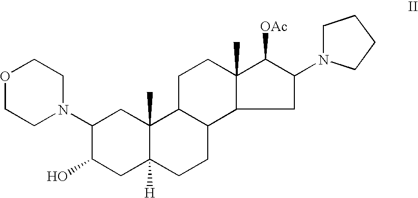

The above patent describes the physical and chemical characteristics of rocuronium bromide, but does not detail the identity and quantity of the impurities, the chemical stability, solid state stability, and “shelf life” of the product. Moreover, patent U.S. Pat. No. 4,894,369 does not disclose whether it is possible to provide rocuronium bromide as a stable, pure solid. Furthermore, no information is provided in relation to how this compound may be obtained in such a form.

The chemical stability, solid state stability, and “shelf life” of a product, and particularly a drug are very important factors. The drag substance should be capable of being effectively stored over appreciable periods of time, without exhibiting a significant change in the active component's physico-chemical characteristics (e.g. its chemical composition, purity, density, hygroscopicity and solubility).

This will enable to separate the chemical process from the pharmaceutical process Additionally, this will also make it possible to carry out each process at different locations and at different times.

The applicants have prepared solid rocuronium bromide by reproducing the process described in the '369 patent. It has been found that the solid rocuronium bromide obtained by the above process, or by using alternative precipitating solvents, contains considerable amounts of residual organic solvents that are higher than pharmaceutically acceptable levels and that are not completely removed from the product during the manufacturing process. Table 1 lists typical amounts of several solvents trapped in the solid rocuronium bromide.

TABLE 1

| Solvent | Amount trapped (%) |
|---|---|
| Methyl acetate | 0.3-0.5 |
| Ethyl acetate | 1.5-3.5 |
| Isopropyl acetate | 1.0 |
| Isobutyl acetate | 4-6 |
| Diethyl ether | 0.1-1.5 |
| Diisopropyl ether | 3.5-4 |
| Methyl t-butyl ether | 6 |
| Dichloromethane | 0.2-0.5 |

Residual organic solvents found in bulk pharmaceutical products are normally removed by drying the bulk in an oven or by blowing the bulk dry on a filter.

Attempts to remove the residual organic solvents to an acceptable level, which is with accordance to pharmaceutical quality, by performing techniques known in the art, failed to reach the desired results. Moreover, attempts to remove the residual organic solvents to the acceptable level by performing more drastic techniques known in the art, such as drying under high vacuum at elevated temperatures for extended periods of time, also failed. The reason is the significant increase in the amounts of impurities in the product, which was probably caused by degradation.

An alternative method of removing the organic solvent using a solution of rocuronium bromide in water did not give a satisfactory result. While freeze drying of the aqueous solution did reduce the isobutyl acetate level to an acceptable level of 0.26%, the impurity levels rose to 0.33%. We have found that rocuronium bromide in water decomposes at room temperature at the rate of 0.25-0.5% in 24 hours. This inherent instability in aqueous solution may explain thc observed rise in the impurities level in our hands.

The present invention overcomes the problems and difficulties associated with the state of the art methods of removing organic solvent residues by providing a process that is effective under very mild conditions and that displaces solvent molecules trapped within the rocuronium bromide.

Accordingly, the applicants have surprisingly found that by spray drying or freeze drying an aqueous solution of rocuronium bromide in the presence of sodium acetate at pH of about 3-5 a stabilized product is obtained, which is suitable as a raw material for producing rocuronium bromide injections.

### SUMMARY OF THE INVENTION

In one aspect, the present invention relates to an improved process for the preparation of a stable solid comprising rocuronium bromide.

In another aspect the present invention relates to an improved process for the preparation of a stable solid comprising substantially pure rocuronium bromide.

In another aspect, the present invention relates to an improved process for the preparation of a stable solid comprising substantially pure rocuronium bromide in high yield.

In another aspect, the present invention relates to an improved process for preparing a stable solid comprising substantially pure rocuronium bromide, which is substantially non-hygroscopic, because when subjected to hygroscopisity test at relative humidity of about 45%, a sample containing 3.1% acetic acid absorbed only about 2% water within 24 hours.

In another aspect, the present invention relates to an improved process for preparing a stable solid comprising substantially pure rocuronium bromide substantially free of impurities.

In another aspect, the present invention relates to an improved process for preparing a stable solid comprising substantially pure rocuronium bromide substantially free of residual organic solvent(s).

In another aspect, the present invention relates to an improved process for the preparation of a stable solid comprising substantially pure rocuronium bromide substantially free of both impurities and of residual organic solvent(s).

In another aspect, the present invention relates to an improved process for preparing a stable, powdered, non-hygroscopic solid comprising substantially pure rocuronium bromide, comprising the steps of: 1. Reacting (2β,3α,5α,16β,17β)-2-(4-morpholinyl)-16(1-pyrrolidinyl)-androstane-3,17-diol, 17-acetate with an excess of allyl bromide in the presence of a suitable solvent; 2. Pouring the reaction mixture to a stirred anti-solvent; 3. Isolating the wet precipitated product in a pure form; 4. Drying the product; 5. Dissolving the product in a buffered aqueous solution; 6. Removing the volatiles from the solution; and 7. Collecting the dry product.

By drying, in the context of the present invention, it is meant removing the organic volatiles by one of the known in the art drying technologies including vacuum ovens, tray ovens, rotary ovens and fluidized bed dryers.

By removing the volatiles, in the context of the present invention, it is meant removing organic and inorganic volatiles by one of the known in the art methods including spray-drying and freeze-drying.

In another aspect, the present invention relates to an improved process for the preparation of a stable solid comprising substantially pure rocuronium bromide and sodium acetate in amount that is in the range of from 10% to 25% w/w, more preferably in the range of from 15% to 20% w/w and most preferably in the range of from 18% to 20% w/w, in respect to the total weight of the product.

In another aspect, the present invention relates to an improved process for preparing a stable, dry, powdered and substantially pure rocuronium brormde that is suitable as a raw material for producing rocuronium bromide injections.

The present invention deals also with processes for the purification of impure rocuronium bromide.

By impure rocuronium bromide, in this context, it is meant a product, which is not with accordance to pharmaceutical quality, that is a product containing impurities, such as starting materials, catalyst components, by-products or residual organic solvent(s) in amounts higher than the allowed pharmaceutical level, with respect to the total weight of the product.

By residual organic solvent(s), in this context, it is meant a solvent or solvents that are trapped in the solid product and are not completely removed from the product during the manufacturing process.

By substantially free, in this context, it is meant a product having impurities and/or residual organic solvent(s), as defined hereinbefore, in accordance with the pharmaceutically acceptable level.

In yet another aspect, the present invention relates to an improved process for the purification of impure rocuronium bromide

In another aspect, the present invention relates to improved process for the purification of impure rocuronium bromide in high yield.

In another aspect, the present invention relates to improved process for the purification of impure rocuronium bromide characterized in that the product is substantially free of impurities.

In another aspect, the present invention relates to improved process for the purification of impure rocuronium bromide characterized in that the product is substantially free of residual organic solvent(s).

In another aspect, the present invention relates to improved process for the purification of impure rocuronium bromide characterized in that the product is substantially free of both impurities and of residual organic solvent(s).

In yet another aspect, the present invention relates to a process for obtaining a stable, powdered, non-hygroscopic solid comprising substantially pure rocuronium bromide, the process comprising the steps of: 1. Dissolving impure rocuronium bromide in a suitable solvent; 2. Pouring the reaction mixture to a stirred anti-solvent; 3. Isolating the wet precipitated product; 4. Drying the product; 5. Dissolving the product in a buffered aqueous solution; 6. Removing the volatiles from the solution; and 7. Collecting the dry product.

In yet another aspect, the present invention relates to a process for obtaining a stable, powdered, non-hygroscopic solid comprising substantially pure rocuronium bromide, the process comprising the steps of: 1. Dissolving impure rocuronium bromide in a suitable solvent; 2. Drying, spray-drying or lyophilizing the product; 3. Dissolving the product in a buffered aqueous solution; 4. Removing the volatiles from the solution; and 5. Collecting the dry product.

In yet another aspect, the present invention relates to a process for obtaining a stable, powdered, non-hygroscopic solid comprising substantially pure rocuronium Bromide, the process comprising the steps of: 1. Suspending impure rocuronium bromide in a suitable anti-solvent; 2. Isolating the precipitated product in a pure form; 3. Drying the product; 4. Dissolving the product in a buffered aqueous solution; 5. Removing the volatiles from the solution; and 6. Collecting the dry product.

In yet another aspect, the present invention relates to a process for obtaining a rocuronium bromide comprising the steps of: 1. Dissolving impure rocuronium bromide in a buffered aqueous solution; 2. Removing the volatiles from the solution; and 3. Collecting the dry product.

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The present invention meets a need in the art for improved processes for the preparation and for the purification of rocuronium bromide in high purity and yield.

The present invention meets a need in the art for improved processes for the preparation and for the purification of rocuronium bromide, suitable for use in pharmaceuticals in high purity and in high yield.

In accordance with the present invention, an improved process for preparing a stable, powdered solid comprising substantially pure rocuronium bromide having the structure of Formula I in a high yield is provided. The process comprises 1. Reacting (2β,3α,5α,16β,17β)-2-(4-morpholinyl)-16(1-pyrrolidinyl)-androstane-3,17-diol, 17-acetate with an excess of allyl bromide in the presence of a suitable solvent; 2. Pouring the reaction mixture to a stirred anti-solvent; 3. Isolating the wet precipitated product in a pure form; 4. Drying the product; 5. Dissolving the product in a buffered aqueous solution; 6. Removing the volatiles from the solution; and 7. Collecting the dry product.

In one embodiment of the present invention, step (1) is carried out in the presence of an organic solvent.

In a preferred embodiment of the present invention, step (1) is carried out in an organic solvent As used herein, the term “solvent” refers to a single compound or a mixture of compounds. The term “organic solvent” means a solvent conventionally understood as such in the art, including a solvent in which non-polar or hydrophobic compounds are preferentially and substantially soluble.

Non limiting examples of organic solvents usable in context of the present invention include halogenated hydrocarbons, acetonitrile, N,N-dimethylformamide, N,N-dimethylacetamide, dimethylsulfoxide, and the like and mixtures thereof

In a preferred embodiment of the present invention, step (1) is carried out in halogenated hydrocarbons or in acetonitrile, more preferably in dichloromethane or in acetonitrile or in any mixture thereof.

In another embodiment of the present invention, step (1) is conducted at a temperature in the range of from about 10° C. to about 50° C., more preferably from about 15° C. to about 30° C., most preferably at an ambient temperature.

In another embodiment of the present invention, allyl bromide is added in an excess ranges from 5-fold to 30-fold relative to (2β,3α,5α,16β,17β)-2-(4-morpholinyl)-16(1-pyrrolidinyl)-androstane-3, 17-diol,17-acetate, more preferably from 10-fold to 20-fold, most preferably of about 17-fold.

In yet another embodiment of the present invention, the mixture of step (1) is poured into a stirred, preferably cold, anti-solvent in such a way so as to result in precipitation. The term “anti-solvent” is defined as any solvent in which the rocuronium bromide is poorly soluble.

Non-limiting examples of anti-solvents usable in context of the present invention include alkyl acetates, dialkyl ethers, wherein the dialkyl groups are the same or different, and low boiling point hydrocarbons and mixtures thereof

As used herein, the term “alkyl” refers to a saturated aliphatic hydrocarbon including straight chain and branched chain groups. Preferably, the alkyl group has 1 to 10 carbon atoms. Whenever a numerical range; e.g., “1-10”, is stated herein, it means that the group, in his case the alkyl group, may contain 1 carbon atom, 2 carbon atoms, 3 carbon atoms, etc., up to and including 10 carbon atoms. More preferably, it is a medium size alkyl having 1 to 7 carbon atoms. Most preferably, it is a lower alkyl having 1 to 5 carbon atoms.

As used herein, the term “low boiling point hydrocarbons” refers to a saturated or unsaturated aliphatic hydrocarbon including straight chain and branched chain groups. Preferably, the hydrocarbon has 5 to 10 carbon atoms. Whenever a numerical range; e.g., “5-10”, is stated herein, it means that the hydrocarbon, may contain 5 carbon atoms, 6 carbon atoms, 7 carbon atoms, etc., up to and including 10 carbon atoms. Most preferably, it is a medium size hydrocarbon having 5 to 7 carbon atoms.

Representative examples of anti-solvents that are usable in the context of the present invention include, without limitation, methyl acetate, ethyl acetate, isopropyl acetate, isobutyl acetate, methyl t-butyl ether, diisopropyl ether, diethyl ether, pentane, hexane, heptane, petroleum ethers and mixtures thereof.

In yet another embodiment of the present invention, the product is isolated from the reaction mixture of step (2) by filtration or centrifugation. The product thus obtained may be treated with a washing solution containing an anti-solvent as defined above.

In yet another embodiment of the present invention, in step (4) the isolated product of step (3) can be dried using conventionally known methods to give rocuronium bromide substantially free of impurities, which may contain pharmaceutically unacceptable levels of residual organic solvent(s).

The drying stage may be carried out by increasing the temperature or reducing the pressure or a combination of both. Non limiting examples of drying technologies or equipments usable in context of the present invention include vacuum ovens, tray ovens, rotary ovens and fluidized bed dryers.

In yet another embodiment of the present invention, in step (5) the isolated product of step (4) is dissolved in a buffered aqueous solution. The buffered aqueous solution can be prepared by dissolving sodium acetate (anhydrous or trihydrate) and acetic acid in water. The amount of the sodium acetate is between 5 and 30 parts in respect to the amount of the rocuronium bromide, more preferably the amount of the sodium acetate is between 15 and 20 parts in respect to the amount of the rocuronium bromide. The pH of the buffered aqueous solution is in the range of from 2 to 6, more preferably in the range of from 3 to 5 and most preferably in the range of from 4 to 4.5.

In another embodiment of the present invention, step (5) may be conducted at a temperature in the range of from about 10° C. to about 50° C., more preferably about 15° C. to about 30° C., most preferably at an ambient temperature.

In another embodiment of the present invention, the solution of step (5) is further dried and the product is collected using conventional methods to give substantially pure rocuronium bromide in high yield. Non-limiting examples of the conventional drying methods usable in context of the present invention include spray-drying and freeze-drying.

In yet another embodiment of the present invention, steps 6-7 are preferably conducted in the dark and in the absence of oxygen.

The product obtained by the process described hereinabove, further contains sodium acetate in amount that is in the range of from 10% to 25% w/w, more preferably in the range of from 15% to 20% w/w and most preferably in the range of from 18% to 20% w/w, in respect to the total weight of the product.

“Substantially pure rocuronium bromide” refers to a product containing rocuronium bromide and impurities in an amount lower than about 0.5% w/w and preferably lower than about 0.1% w/w, in respect to the total weight of the product.

The yield of the process is an important feature of the invention. As described in the examples, rocuronium bromide can be obtained in a yield of over 90%, more preferably over 91%, more preferably over 92%, more preferably over 93%, more preferably over 94%, more preferably over 95%, more preferably over 96%, more preferably over 97%, more preferably over 98%, more preferably over 99% and most preferably quantitatively with respect to the starting amount of the molecule having the structure formula (II).

The above preparation process results in a stable, dry, powdered and non-hygroscopic substantially pure rocuronium bromide that is suitable as a raw material for producing rocuronium bromide injections.

In accordance with the present invention, the first improved purification process for obtaining a stable, powdered solid comprising substantially pure rocuronium bromide in a high yield, comprises: 1. Dissolving impure rocuronium bromide in a suitable solvent; 2. Pouring the reaction mixture to a stirred anti-solvent: 3. Isolating the wet precipitated product, 4. Drying the product; 5. Dissolving the product in a buffered aqueous solution; 6. Removing the volatiles from the solution; and 7. Collecting the dry product.

In accordance with the present invention, the second improved purification process for obtaining a stable, powdered solid comprising substantially pure rocuronium bromide having the structure of Formula I in a high yield comprises: 1. Dissolving impure rocuronium bromide in a suitable solvent; 2. Drying, spray-drying or lyophilizing the product; 3. Dissolving the product in a buffered aqueous solution; 4. Removing the volatiles from the solution; and 5. Collecting the dry product.

In accordance with the present invention, the third improved purification process for obtaining a stable, powdered solid comprising substantially pure rocuronium bromide having the structure of Formula I in a high yield comprises: 1. Suspending impure rocuronium bromide in a suitable anti-solvent; 2. Isolating the precipitated product in a pure form; 3. Drying the product; 4. Dissolving the product in a buffered aqueous solution; 5. Removing the volatiles from the solution; and 6. Collecting the dry product.

In accordance with the present invention, the fourth improved purification process for obtaining a stable, powdered solid comprising substantially pure rocuronium bromide having the structure of Formula I in a high yield comprises: 1. Dissolving impure rocuronium bromide in a buffered aqueous solution; 2. Removing the volatiles from the solution; and 3. Collecting the dry product.

The impure rocuronium bromide as used herein refers to a rocuronium bromide isolated from any process conventionally known in the art or to be developed in the future.

The above purification processes results in a stable, dry, powdered substantially pure rocuronium bromide that is suitable as a raw material for producing rocuronium bromide injections

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

Analysis of rocuronium bromide By High Performance Liquid Chromatography (HPLC):

High performance liquid chromatography (“HPLC”) was performed using the following conditions: Column and packing—Hypersil Silica 5μ250×4.6 mm, Thermo Hypersil-Keystone, P.N. 30005-254630; TV detection—UV operated at 210 nm; flow rate: 2 ml/min; Mobile phase: Buffer:. Acetonitrile=1:9 (v/v); Buffer preparation. Weighing 4.53 g of Tetramethylammonium Hydroxide Pentahydrate into 1000 ml volumetric flask. Dissolving and completing the volume with water and adjusting the pH to 7.4 with 85% Phosphoric Acid; Injection volume: 5 μL; Run time: 2.5 times the retention time of rocuronium bromide.

Analysis of rocuronium bromide by Gas Chromatography (GC):

Instrument:

Agilent 6890 Series GC system, equipped with an FID detector and a split mode injector and PAL head space device.

Column:

DB-624, 30 m, ID=0.53 mm, film thickness 3 μm (J&W CN 125-1334 is suitable)

Temperature Programming:

| | |
|---|---|
| Initial oven temperature: | 40° C. |
| Hold time: | 10 min. |
| Program 1 final oven temperature: | 130° C. |
| Heating rate: | 12° C./min. |
| Program 2 final oven temperature: | 250° C. |
| Heating rate: | 50° C./min |
| Final hold time | 6 min |
| Detector (FID) temperature: | 250° C. |
| Injector temperature: | 220° C. |
| Carrier gas: | Helium |
| Nominal initial flow: | 3.7 mL/min |
| Injector | |
| Split ratio 1:25 | |

Conditions for head space injector:

| | |
|---|---|
| Injection volume: | 1000 μL |
| Incubation temperature: | 80° C. |
| Incubation time: | 10 minutes |
| Agitator speed: | 500 rpm |
| Syringe temperature: | 125° C. |
| Fill speed: | 300 μL/sec |
| Fill strokes | 0 |
| Pullup delay | 0 |
| Injection speed: | 1000 μL/sec |
| Post injection delay: | 200 ms |
| Syringe flushing time: | 300 sec |
| Cycle runtime: | 34 minutes |

Spray-Drying was Performed by:

Mini spray dryer model Buchi B-190 was used for spray drying. System description: Heater 1.8 KW, Temperature range: 40-220° C., Evaporation Rate: approx 1500 ml/hour.

Freeze-Drying was Performed by:

VirTis AdVantage single shelf freeze-dryer with shelf temperatures that ranges from −70° C. to +60° C., with process condenser temperatures of −85° C.

### Example 1

A mixture of (2β,3α,5α,16β,17β)-17-acetoxy-3-hydroxy-2-(4-morpholynyl)-16-(1-pyrrolidinyl)androstane-3,17-diol, 17-acetate ( Compound II, 10 grams) allyl bromide-(30 ml) and acetonitrile (40 ml) was stirred at room temperature for 3 hours. The solution was gradually poured to a vigorously stirred isobutyl acetate (480 ml). The precipitated rocuronium bromide was filtered.

HPLC analysis of the product showed that it contained 0.15% of total impurities.

GC analysis of the product showed that it contained 5.7% isobutyl acetate. Acetonirtile was not detected.

### Example 2

A mixture of (2β,3α,5α,16β,17β)-17-acetoxy-3-hydroxy-2-(4-morpholynyl)-16-(1-pyrrolidinyl)androstane-3,17-diol, 17-acetate ( Compound II, 10 grams) allyl bromide (30 ml) and acetonitrile (40 ml) was stirred at room temperature for 3 hours. The solution was gradually poured to a vigorously stirred ethyl acetate (480 ml). The precipitated rocuronium bromide was filtered.

### Example 3

A mixture of (2β,3α,5α,16β,17β)-17-acetoxy-3-hydroxy-2-(4-morpholynyl)-16-(1-pyrrolidinyl)androstane-3,17-diol, 17-acetate ( Compound II, 5 grams) allyl bromide (13 ml) and acetonitrile (20 ml) was stirred at room temperature for 3 hours. The solution was gradually poured to a vigorously stirred diethyl ether (120 ml).The precipitated rocuronium bromide was filtered.

HPLC analysis of the product showed that it contained 0.35% of total impurities.

GC analysis of the product showed that it contained 0.66% diethyl ether and 0.17% acetonirtile.

### Example 4

Rocuronium bromide prepared according to the procedures outlined in examples 1 and 2 was subjected to drying under different conditions. The dried products were analyzed for impurities and solvent content, as depicted in Table 2.

TABLE 2

| | | | Total | |
|---|---|---|---|---|
| | | | impurities | Solvent |
| No. | Solvent | Drying conditions | (%) | content (%) |
| 1 | Isobutyl acetate | Vacuum oven, | 0.21 | 4.30 |
| | | 40° C., overnight | | |
| 2 | Isobutyl acetate | Vacuum oven, | 0.10 | 6.20 |
| | | 40° C., overnight | | |
| 3 | Isobutyl acetate | Entry 1, then | 0.34 | 3.54 |
| | | fluidized bed drying | | |
| | | 40° C., 6 hours | | |
| 4 | Isobutyl acetate | Vacuum oven, | 0.63 | 4.72 |
| | | 60° C., overnight | | |
| 5 | Isobutyl acetate | Vacuum oven, | 0.51 | 5.11 |
| | | 60° C., overnight | | |
| 6 | Ethyl acetate | Vacuum oven, | 0.67 | 3.00 |
| | | 60° C., overnight | | |
| 7 | Diethyl ether | Vacuum oven, | 0.35 | 0.66 |
| | | 60° C., overnight | | |

### Example 5

Rocuronium bromide (2 grams) prepared according to example 1 and dried in a manner similar to entries 1 and 2 in table 2 (see example 4), was dissolved in degassed water (30 ml) and freeze dried. The product was obtained as a fluffy, highly hygroscopic powder.

HPLC analysis of the product showed that it contained 0.33% of total impurities.

GC analysis of the product showed that it contained 0.26% isobutyl acetate.

### Example 6

Rocuronium bromide (2 grams) prepared according to example 1 and dried in a manner similar to entries 1 and 2 in table 2 ( see example 4), was dissolved in degassed water and acetic acid (˜0.5 ml) was added to bring the pH to about 5. The solution was freeze dried. The product was obtained as very thick syrup.

HPLC analysis of the product showed that it contained 0.10% of total impurities.

GC analysis of the product showed that it contained 0.72% isobutyl acetate. Acetonirtile was not detected.

### Example 7

Acetate buffer (pH 4.5) was prepared by combining anhydrous sodium acetate (5.4 grams), acetic acid (2.4 grams) and water (to 100 ml).

Rocuronium bromide (2 grams) prepared according to example 1 and dried in a manner similar to entries 1 and 2 in table 2 (see example 4), was dissolved in the above buffer (7.4 ml) and degassed water (30 ml). The mixture was freeze dried. The product was obtained as a powder.

HPLC analysis of the product showed that it contained 0.06% of total impurities.

GC analysis of the product showed that it contained 0.08% of isobutyl acetate.

### Example 8

In a similar manner material precipitated from ethyl acetate (see example 4, table 2, entry 6) was subjected to the treatment as described in example 7.

HPLC analysis of the product showed that it contained 0.06% of total impurities.

GC analysis of the product showed that it contained 0.04% of ethyl acetate.

### Example 9

A solution prepared according to the procedure outlined in example 7 was spray dried (inlet temperature 120° C., outlet temperature ˜78° C.). The product was obtained as a flowing powder.

HPLC analysis of the product showed that it contained 0.06% of total impurities.

GC analysis of the product showed that it contained 0.0008% of isobutyl acetate.

### Example 10

To a solution of rocuronium bromide (6 gram) prepared in a similar way as described in example 1, in water (90 ml) was added acetate buffer (20 ml, prepared according to example 7). The solution obtained was extracted with n-pentane (15 ml) and the organic phase was discarded. The solution was spray dried.

HPLC analysis of the product showed that it contained 0.12% of total impurities.

GC analysis of the product showed that it contained organic solvents as follows; acetonitrile—not detected, pentane—49 ppm, isobutyl acetate—not detected.

What is claimed is:

1. A process for preparing a stable, powdered solid comprising substantially pure rocuronium bromide, the process comprising:
i. reacting (2β,3α,5α,16β, 17β)2-(4-morpholinyl)-16(1-pyrrolidinyl)-androstane-3,17-diol, 17-acetate with an excess of allyl bromide in the presence of a suitable solvent;
ii. pouring the reaction mixture to a stirred anti-solvent;
iii. isolating the wet precipitated product in a pure form;
iv. drying the product,
v. dissolving the product in a buffered aqueous solution;
vi. removing the volatiles from the solution; and
vii. collecting the dry product.

2. A process according to claim 1 wherein said solvent is an organic solvent.

3. A process according to claim 2, wherein said organic solvent is selected from a group consisting of halogenated hydrocarbons, acetonitrile, N,N-dimethylformamide, N,N-dimethylacetamide, dimethylsulfoxide, and the like and mixtures thereof.

4. A process according to claim 3, wherein said organic solvent is dichloromethane, acetonitrile or any mixture thereof.

5. A process according to claim 1, wherein said process is conducted at a temperature in the range of from about 10° C. to about 50° C., more preferably from about 15° C. to about 30° C., most preferably at an ambient temperature.

6. A process according to claim 1, wherein said allyl bromide is added in an excess ranges from 5-fold to 30-fold relative to (2β,3α,5α,16β,17β)-2-(4-morpholinyl)-16(1-pyrrolidinyl)-androstane-3,17-diol, 17-acetate, more preferably from 10-fold to 20-fold, most preferably of about 17-fold.

7. A process according to claim 1, wherein said anti-solvent is selected from a group consisting of alkyl acetates, dialkyl ethers, wherein the dialkyl groups are the same or different, and low boiling point hydrocarbons and mixtures thereof

8. A process according to claim 7, wherein said anti-solvent is selected from a group consisting of methyl acetate, ethyl acetate, isopropyl acetate, isobutyl acetate, methyl t-butyl ether, diisopropyl ether, diethyl ether, pentane, hexane, heptanes, petroleum ethers and mixtures thereof.

9. A process according to claim 1, wherein said isolating is performed by filtration or centrifugation.

10. A process according to claim 1, wherein said drying stage is carried out by increasing the temperature or reducing the pressure or a combination of both.

11. A process according to claim 10, wherein said drying of the product is carried out by any of the technologies or equipments selected from a group consisting of vacuum ovens, tray ovens, rotary ovens and fluidized bed dryers.

12. A process according to claim 1, wherein said aqueous solution is prepared by dissolving sodium acetate (anhydrous or trihydrate) and acetic acid in water.

13. A process according to claim 12, wherein said amount of sodium acetate ranges between 5 and 30 parts in respect to the amount of the rocuronium bromide, more preferably the amount of the sodium acetate ranges between 15 and 20 parts with respect to the amount of the rocuronium bromide.

14. A process according to claim 12, wherein a pH of said buffered aqueous solution is in the range of from 2 to 6, more preferably in the range of from 3 to 5 and most preferably in the range of from 4 to 4.5.

15. A process according to claim 1, wherein said step (v) is conducted at a temperature in the range of from about 10° C. to about 50° C., more preferably from about 15° C. to about 30° C., most preferably at an ambient temperature.

16. A process according to claim 1, wherein step (vi) is carried out using any of the technologies selected from a group comprising of spray-drying and freeze-drying.

17. A process according to claim 1, wherein said removing the volatiles from solution (step vi) and collecting the dry product (step vii) are preferably conducted in the dark and in the absence of oxygen.

18. A process according to claim 1, wherein said stable solid further comprising sodium acetate in amount that ranges from 10% to 25% w/w, more preferably from 15% to 20% wIw and most preferably from 18% to 20% w/w, respect to the total weight of the product.

19. A process according to claim 1, wherein said stable solid comprising rocuronium bromide and impurities in an amount lower than about 0.5% w/w and preferably lower than about 0.1% w/w, with respect to the total weight of the product.

20. A process according to claim 1, wherein said substantially pure rocuronium bromide is obtained in a yield of over 90%, more preferably over 91%, more preferably over 92%, more preferably over 93%, more preferably over 94%, more preferably over 95%, more preferably over 96%, more preferably over 97%, more preferably over 98%, more preferably over 99% and most preferably quantitatively with respect to the starting amount of the molecule having the structure formula (II).

21. A process according to claim 1, wherein said stable solid comprising substantially pure rocuronium bromide is suitable as a raw material for producing rocuronium bromide injections.

22. A process for obtaining a stable, powdered solid containing substantially pure rocuronium bromide, the process comprising:
i. dissolving impure rocuronium bromide in a suitable solvent;
ii. pouring the reaction mixture to a stirred anti-solvent;
iii. isolating the wet precipitated product;
iv. drying the product;
v. dissolving the product in a buffered aqueous solution;
vi. removing the volatiles from the solution; and
vii. collecting the dry product.

23. A process for obtaining a stable, powdered solid containing substantially pure rocuronium bromide, the process comprising:
i. dissolving impure rocuronium bromide in a suitable solvent;
ii. drying, spraydrying or lyophilizing the product;
iii. dissolving the product in a buffered aqueous solution;
iv. removing the volatiles from the solution; and
v. collecting the dry product.

24. A process for obtaining a stable, powdered solid containing substantially pure rocuronium bromide, the process comprising:
i. suspending impure rocuronium bromide in a suitable anti-solvent;
ii. isolating the precipitated product in a pure form;
iii. drying the product;
iv. dissolving the product in a buffered aqueous solution;
v. removing the volatiles from the solution; and
vi. collecting the dry product.

25. A process for obtaining a stable, powdered solid containing substantially pure rocuronium bromide, the process comprising:
i. dissolving impure rocuronium bromide in a buffered aqueous solution;
ii. removing the volatiles from the solution; and
iii. collecting the dry product.

26. A stable powdered solid comprising substantially pure rocuronium bromide, having purity equal to or greater than 99.5%.

27. A stable solid according to claim 26, being substantially free of residual organic solvent(s).

28. A stable solid according to claim 26, comprising rocuronium bromide and impurities in an amount lower than about 0.5% w/w and most preferably lower than about 0.1% w/w, with respect to the total weight of the product.

29. A stable solid according to claim 26, suitable as a raw material for producing rocuronium bromide injections.

30. A stable solid according to claim 26, comprising rocuronium bromide absorbing about 2% water within 24 hours at relative humidity of about 45%.

31. A stable solid comprising substantially pure rocuronium bromide containing sodium acetate in an amount that ranges from 10% to 25% w/w, more preferably from 15% to 20% w/w and most preferably from 18% to 20% wlw, with respect to the total weight of thc product.
